(19) United States
(12) Patent Application Publication
(10) Pub. No.: US 2022/0072186 A1
(43) Pub. Date: Mar. 10, 2022

(54) Disinfecting Circadian Lighting Device

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/015,367

(22) Filed: Sep. 9, 2020

(51) Int. Cl. A61L 9/20; A61L 2/24; A61L 2/20

(52) U.S. Cl. CPC A61L 9/20; A61N 2005/0652; A61L 2/20; A61L 2/24

(57) ABSTRACT

A disinfection circadian lighting device includes a housing and two means of lighting. The first means of lighting is a general lighting means and it includes a first light source, a first lens, and a first driver. The second means of lighting is a germicidal lighting means and it includes a second light source, a second lens, and a second driver. The second light source is a far UVC light source with its spectral power distribution mainly in the wavelength range 200 nm to 230 nm. The first light source may comprise a third light source and a fourth light source, where third light source is a blue-depleted light source and the fourth light source is a blue-enriched light source. A circadian controller can mix the light output of the third and the fourth light sources according to a circadian schedule.

### BACKGROUND

### Technical Field

The present disclosure pertains to the field of lighting devices and, more specifically, proposes a disinfecting circadian lighting device.

### Description Of Related Art

It is known that circadian lighting fixtures that provide a blue-enriched light during daytime and a blue-depleted light during nighttime could help regulate the circadian rhythm of the user and improve user's sleep quality in particular and health in general. However, the circadian lighting fixtures have no effect against pathogens in the surrounding area. In contrast, UVC based germicidal lighting is known to be effective in disinfecting against pathogens in air or on a surface. However, UVC is also known to cause skin and eye damages to the user. As a result, UVC based germicidal lighting device is recommended to be turned on when no users are nearby, or that the UVC light source is sealed inside a metal compartment. One recent study by Columbia University demonstrates that a far UVC light source at 222 nm is effective in killing bacteria and viruses, but without the side effects of causing skin or eye damages (http://www.columbia.edu/˜djb3/papers/Germicidal%20Efficacy%20and%20Mammalian %20Skin%20Safety%20of%20222-nm%20UV%20Light.pdf). Moreover, a follow-up study shows the 222 nm far UVC light can efficiently and safely inactivates airborne human coronaviruses (https://www.nature.com/articles/s41598-020-67211-2).

The present disclosure proposes a disinfecting circadian lighting device that combines the functionality of circadian lighting and germicidal lighting to reap the benefits of both, while addressing the requirements on energy efficiency and operation safety.

### SUMMARY

In one aspect, the disinfecting circadian lighting device comprises a housing, a first light source, a second light source, a first lens, a second lens, a first driver, a second driver, a first means of lighting, and a second means of lighting. The housing houses the first light source, the second light source, the first lens, the second lens, the first driver, and the second driver. The first light source emits a first light with a wavelength in a range of 400 nm to 700 nm, and the first light source emits the first light through the first lens. The second light source emits a second light with a wavelength in a range of 200 nm to 230 nm, and the second light source emits the second light through the second lens. The first means of lighting is a general lighting means and it includes the first light source, the first lens, and the first driver for converting an external power to an internal power suitable for activating the first light source. The second means of lighting is a germicidal lighting means and it includes the second light source, the second lens, and the second driver for converting an external power to an internal power suitable for activating the second light source. The use of the second lens on the second light source is critical to the germicidal lighting means. Firstly, the second light source may be activated by a high voltage. The use of the second lens provides a safety guard over the high-voltage used by the second source. Secondly, the second lens may use to filter the second light to achieve the desirable 200 nm-230 nm wavelength range at a much lower cost, as compared to the cost of using a second light source with a natural 200 nm-230 nm wavelength range. Thirdly, the second light source may comprise a rare gas and thus needs to be contained in the second lens.

The first means and the second means of lighting may be turned on and off simultaneously through a single on/off switch. There are situations when a user may want to turn on/off the first means and the second means of lighting independently, for example, turning off the first means of lighting during off-hours for energy savings, or turning off the second means of lighting when people are in the room for avoiding far UVC exposure. Therefore, in some embodiments, the first means of lighting and the second means of lighting can be turned on and off independently. With such embodiments, a centralized lighting control system may be configured such that the first means of the lighting and the second means of lighting may operate on two different schedules.

In some embodiments, the device further includes an occupancy senor such that the occupancy sensor turns on the first means of lighting upon a motion detection and turns off the first means of lighting after detecting no motion for a preconfigured time (e.g. 5 minutes), without affecting the operation of the second means of lighting. By incorporating an occupancy sensor for the first means of lighting, the device becomes more energy efficient.

In some embodiments, the device further includes a vacancy senor such that the vacancy sensor turns off the second means of lighting upon a motion detection and turns on the second means of light after detecting no motion for a preconfigured time (e.g. 3 minutes), without affecting the operation of the first means of lighting. With the use of a vacancy sensor for the second means of lighting, it would prevent a user from being exposed to far UVC light.

In some embodiments, the second light source is a rare gas, such as Krypton- Chloride (Kr-Cl) or Krypton-Bromine (Kr-Br) or their combination, and the rare gas is contained inside the second lens. Moreover, the second driver has a means of electric discharge for exciting the rare gas molecule so that the excited rare gas molecule releases its excitation energy in the form of a UV photon in the wavelength range 200 nm to 230 nm. An excited Kr-CI molecule releases its excitation energy with a 222 nm UV photon and an excited Kr-Br molecule releases its excitation energy with a 207 nm UV photon. There are different types of electric discharge. In some embodiments, the second driver has a means of dielectric barrier discharge (DBD). With DBD, a high voltage electrode and the ground electrode are separated by an insulating dielectric barrier.

The manufacturing technology of light emitting diode (LED) can make an LED with any target wavelength. It would be desirable in making an LED emitting a light with a wavelength in a range of 200 nm to 230 nm. Therefore, in some embodiments, the second light source comprises one or more light emitting diodes.

With the present disclosure, the second light source emits a second light with a wavelength in a range of 200 nm to 230 nm. This however does not preclude that the second light may have wavelength outside of the 200 nm to 230 nm range, for example, in the normal and harmful UVC wavelength range from 230 nm to 280 nm. In order to ensure the source light source doesn't emit too much harmful UVC, in some embodiments, the second light source would emit the second light with a spectral power distribution (SPD) greater than 95% in the wavelength range of 200 nm to 230 nm. In other words, the second light will have an SPD less than 5% outside of 200 nm-230 nm range, thus greatly reducing spectral power of the harmful UVC wavelength.

Glass and plastic are two materials used often for lamp lens. However, they would filter out UV wavelength, and thus are not suited for the second lens. In comparison, quartz provides much better transmission of UV light. Therefore, in some embodiments, the second lens comprises quartz.

The cost may be too high to manufacture the second light source with a natural SPD greater than 95% in the 200 nm-230 nm wavelength range. A more cost-effective alternative may be to use an optical filtering to filter out any undesirable wavelength from the second light source. Therefore, in some embodiments, the second lens is a bandpass optical filter and removes any wavelength outside of the wavelength range of 200 nm to 230 nm, so that the light emitted out of the second lens will has a 100% SPD in wavelength range of 200 nm to 230 nm.

In addition to use a bandpass filter as the second lens, it is also possible use another light-filtering medium for filtering the undesirable wavelength from the second light. A light-filtering medium may not be able to block completely the wavelength outside the 200 nm-230 nm wavelength range, like an optical bandpass filter could. However, it would still be useful if it could reduce the SPD of the second light outside the 200 nm-230 nm range. In some embodiments, a light-filtering medium is used on the second light to produce a filtered light with a greater than 95% SPD in the wavelength range of 200 nm to 230 nm. In some embodiments, the light-filtering medium is an optical lens external to the second lens. In some other embodiments, the light-filtering medium is a light-filtering coating on the surface of the second lens. In some other embodiments, the light-filtering medium is a light-filtering coating on the surface of the second light source. There may be more than one layers of light-filtering coating to effectively filtering out all wavelength outside the 200 nm-230 nm wavelength range.

With a long lifetime, the light emitting diode is an obvious choice for the first light source. In some embodiments, the first light source is made of one or more light emitting diodes.

For general lighting, it is preferred that the first means of lighting is continuously dimmable. In some embodiments, the first driver is dimmable and is capable of dimming the first light continuously.

The UVC light source, especially the far UVC light source, has a short lifetime around 6,000 to 8,000 hours. In comparison, a white LED light source has a lifetime 60,000 to 80,000 hours. When a lighting fixture has both types of light source, the lifetime of the fixture would be restricted to the shorter lifetime of these two types of light source. In this case, its lifetime would be determined by the lifetime of the far UVC light source. A lighting fixture that could be used only for 6,000 to 8,000 hours is not a practical product. A fixture is expected to last anywhere from 5 to 10 years. It is therefore critical to extend the lifetime of the far UVC light source for this disinfecting germicidal lighting device to be more useful. One option is to enable the replacement of the far UVC light source when it failed, and ideally without using any tool for performing the replacement. In some embodiments, the second light source is replaceable without using any tool. Moreover, in some embodiments, the housing includes standard electric socket, e.g., G13, G5, R17D, FAB, 2G11, G24Q, etc., and the second light source has a corresponding electric base so the second light source may be inserted into the electric socket on the housing without any tool.

In some embodiments, the first light source is made of a third light source and a fourth light source, and the device further includes a circadian controller that is housed by the housing. The third light source is a blue-depleted light source and emits a third light with a SPD less than 5% in the 410-490 nm wavelength range. The fourth light source is a blue-enriched light source and emits a fourth light with SPD greater than 15% in the 410-490 nm wavelength range. The circadian controller mixes the third light and the fourth light to generate the light output for the first means of lighting.

In some embodiments, the operation of the circadian controller is performed manually by a user. In some other embodiments, the operation of the circadian controller is performed automatically according to a circadian schedule. Here is an example of a 24-hour circadian schedule:
00:00-06:00: the third light source at 100% light output, the fourth light source at 0% light output
06:00-07:00: the third light source at 70% light output, the fourth light source at 30% light output
07:000-8:00: the third light source at 30% light output, the fourth light source at 70% light output
08:00-09:00: the third light source at 10% light output, the fourth light source at 90% light output
09:00-15:00: the third light source at 0% light output, the fourth light source at 100% light output
15:00-16:00: the third light source at 10% light output, the fourth light source at 90% light output
16:00-17:00: the third light source at 30% light output, the fourth light source at 70% light output
17:00-18:00: the third light source at 70% light output, the fourth light source at 30% light output
16:00-00:00: the third light source at 100% light output, the fourth light source at 0% light output

The circadian schedule may be stored inside a smartphone app and sent to the circadian controller via a wireless communication in real time for controlling the circadian controller to change the mixing of the third light and the fourth light. In some embodiments, the circadian controller has a memory module for storing the circadian schedule. In this case, a preconfigured circadian schedule may be stored in the memory module. The circadian schedule may be updated by a smartphone app via a wireless communication.

In some embodiments, the third light source is made of one or more LED, and the fourth light source is made of one or more LED.

### BRIEF DESCRIPTION OF THE DRAWINGS

The accompanying drawings are included to aid further understanding of the present disclosure and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1a schematically depicts a diagram of a disinfecting circadian troffer fixture with a lens covered over the circadian light sources.

FIG. 1b schematically depicts a diagram of the same troffer fixture with the lens over the circadian light sources removed.

FIG. 1c schematically depicts a straight-down view of the same troffer fixture with the lens over the circadian light sources removed.

FIG. 2 schematically depicts a straight-down view of another disinfecting circadian troffer fixture with the lens over the circadian light sources removed.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of germicidal lighting device having different form factors.

The present disclosure discloses a disinfection circadian lighting device includes a housing and two means of lighting. The first means of lighting is a general lighting means and it includes a first light source, a first lens, and a first driver. The second means of lighting is a germicidal lighting means and it includes a second light source, a second lens, and a second driver. The second light source is a far UVC light source with its spectral power distribution mainly in the wavelength range 200 nm to 230 nm. The first light source may comprise a third light source and a fourth light source, where third light source is a blue-depleted light source and the fourth light source is a blue-enriched light source. A circadian controller can mix the light output of the third and the fourth light sources according to a circadian schedule.

### Example Implementations

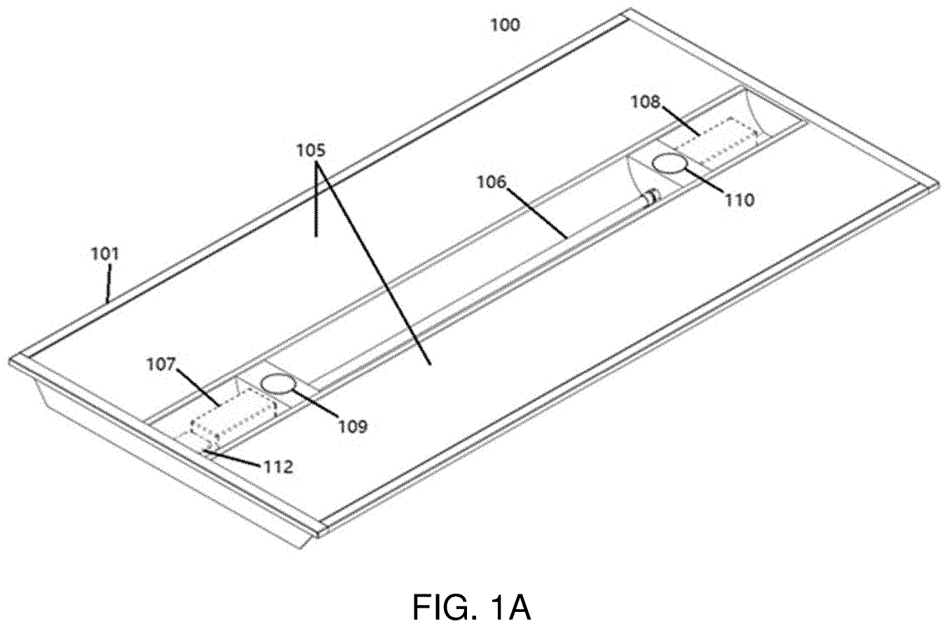

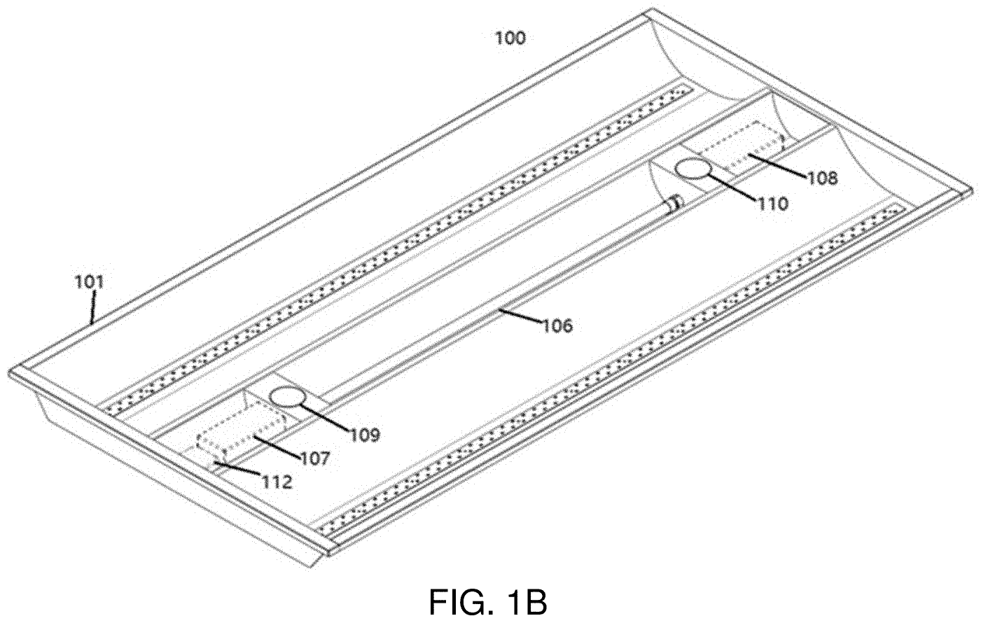

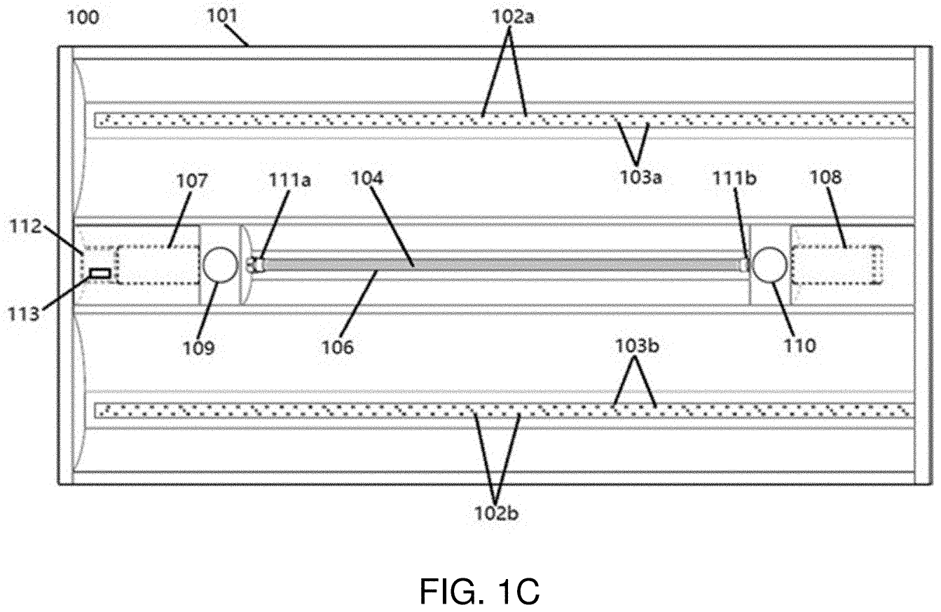

In FIG. 1a-1c, an embodiment of the present disclosure in the form of a 2′×4′ troffer fixture 100. In FIG. 1a, the lens 105 is shown whereas in FIG. 1b, the lens is removed, so that the circadian light sources 102a, 102b, 103a, and 103b can be seen. In FIG. 1c, the same fixture is shown with a straight-down view. The fixture 100 has a housing 101 that houses all other components. The circadian light sources 102a, 102b, 103a, 103b are made of a strip of LED, and they emit lights with a wavelength in a range of 400 nm to 700 nm. Moreover, the light source 102a, 102b is blue-depleted light source and emits a light with an SPD less than 5% in the 410-490 nm wavelength range. The light source 103a, 103b is blue-enriched light source and emits a light with an SPD greater than 15% in the 410-490 nm wavelength range. The light sources 102a, 102b, 103a, 103b emit their lights through the lens 105. The driver 107 converts an external power to an internal power for activating the light sources 102a, 102b, 103a, 103b. The general lighting means of the fixture includes the light sources 102a, 102b, 103a, 103b, the lens 105, the dimmable driver 107, and the occupancy sensor 109. The occupancy sensor 109 will turn on the light sources 102a, 102b, 103a, 103b upon a motion detection, and it will turn off these light sources after detecting no motion for a preconfigured time (e.g., 5 minutes) for energy savings.

The light source 104 is a rare gas, Krypton-Chloride (Kr-Cl), and is contained inside a tubular lens 106. The driver 108 uses dielectric barrier discharge for exciting the Krypton-Chloride molecule so that the excited Krypton-Chloride molecule releases its excitation energy in the form of a 222 nm UV photon, emitting through the lens 106. While the light generated by the Krypton-Chloride molecule is highly concentrated around 222 nm, there may still be a residual wavelength outside the wavelength range of 200 nm to 230 nm, depending on the composition ratio of Krypton-Chloride gas. The lens 106 also functions as a bandpass filter for removing any wavelength outside of the wavelength range of 200 nm to 230 nm.

The germicidal lighting means of the fixture includes the light source 104, the lens 106, the driver 108, and the vacancy sensor 110. The vacancy sensor 110 will turn off the light source 104 upon a motion detection, and it will turn on the light source 104 after detecting no motion for a preconfigured time (e.g., 3 minutes). The motion sensor 109 and the occupancy sensor 110 operates independent of each other, and therefore the turning on/off the light sources 102a, 102b, 103a, 103b and the turning on/off the light source 104 are also independent of each other.

The lens 106 has two G13-based endcaps 111a, 111b, and these two endcaps can be inserted into a pair of G13 socket on the housing 101. Therefore, the replacement of the light source 104 along with the lens 104 and the two G13-based endcaps 111a, 111b can be performed without using any tool.

The driver 107 is dimmable and can dim the light sources 102a, 102b, 103a, 103b continuously. Moreover, a circadian controller 112 can mix the light output of the light sources 102a, 102b, 103a, 103b to generate a combined light output according to a circadian schedule stored in a memory module 113. During daytime, the circadian schedule will generate a blue-enriched light based on the blue-enriched light source 103a, 103b, and during nighttime the circadian schedule will generate a blue-depleted light based on the blue-depleted light source 102a, 102b. During dawn, the circadian schedule will transition from a blue-depleted light to a blue-enriched light, and during dusk from a blue-enriched light to a blue-depleted light.

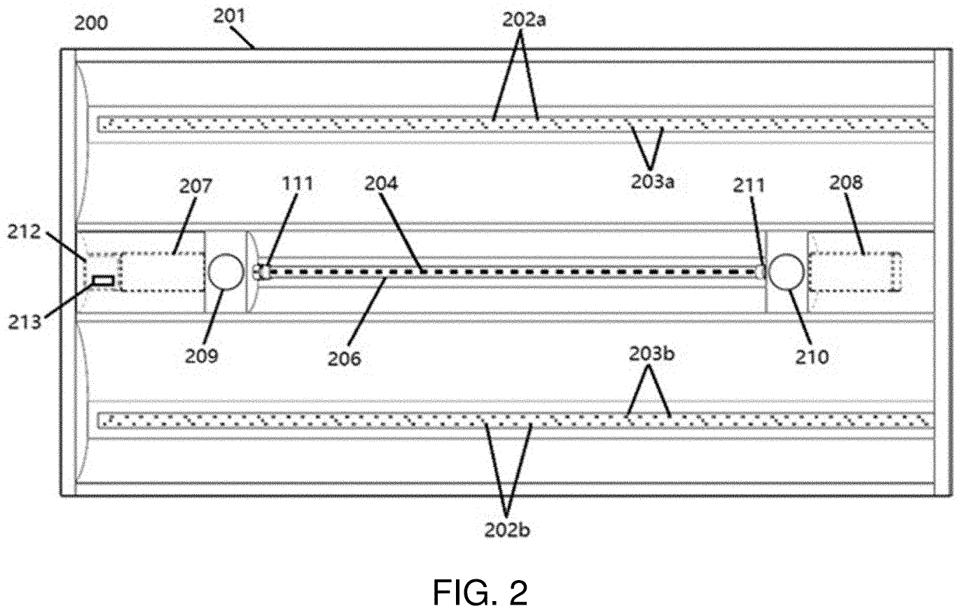

FIG. 2 shows another linear troffer. It has the same construction as the troffer shown in FIG. 1a-1c with the exception that the linear troffer 200 in FIG. 2 uses LED for the germicidal light source 204. An external driver 208 activates the light source 204. It is possible to use an embedded driver inside the lens 206 for activating the LED light source 204. There is a lens for covering the circadian light sources 202a, 202b, 203a, 203b but not shown in FIG. 2.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A disinfecting lighting device, comprising
a housing;
a first light source;
a second light source;
a first lens;
a second lens;
a first driver; and
a second driver,
wherein:
the housing houses the first light source, the second light source, the first lens, the second lens, the first driver, and the second driver,
the first light source is configured to emit a first light with a wavelength in a range of 400 nm to 700 nm,
the second light source is configured to emit a second light with a wavelength in a range of 200 nm to 230 nm,
the first light source is configured to emit the first light through the first lens,
the second light source is configured to emit the second light through the second lens,
the first light source, the first lens, and the first driver together form a first means of lighting, which is a general lighting means, configured to convert an external power to an internal power suitable for activating the first light source, and
the second light source, the second lens, and the second driver together form a second means of lighting, which is a germicidal lighting means, configured to convert an external power to an internal power suitable for activating the second light source.

2. The disinfecting lighting device of claim 1, wherein the first means of lighting and the second means of lighting are configured to be turned on and off independently.

3. The disinfecting lighting device of claim 1, further comprising an occupancy senor configured to turn on the first means of lighting upon a motion detection and turn off the first means of lighting after detecting no motion for a preconfigured time, without affecting an operation of the second means of lighting.

4. The disinfecting lighting device of claim 1, further comprising a vacancy senor configured to turn off the second means of lighting upon a motion detection and turn on the second means of light after detecting no motion for a preconfigured time, without affecting an operation of the first means of lighting.

5. The disinfecting lighting device of claim 1, wherein the second light source comprises a rare gas comprising Krypton-Chloride (Kr-Cl), Krypton-Bromine (Kr-Br) or a combination thereof, wherein the second light source is contained inside the second lens, and wherein the second driver is capable of electric discharge and configured to excite molecules of the rare gas so that an excited rare gas molecule releases an excitation energy as a UV photon in a wavelength range of 200 nm to 230 nm.

6. The disinfecting lighting device of claim 5, wherein a type of electric discharge by the second driver comprises dielectric barrier discharge (DBD).

7. The disinfecting lighting device of claim 1, wherein the second light source comprises one or more light emitting diodes (LEDs).

8. The disinfecting lighting device of claim 1, wherein the second light source is configured to emit the second light with a spectral power distribution (SPD) greater than 95% in a wavelength range of 200 nm to 230 nm.

9. The disinfecting lighting device of claim 1, wherein the second lens comprises quartz.

10. The disinfecting lighting device of claim 1, wherein the second lens comprises a bandpass optical filter configured to remove any wavelength outside of a wavelength range of 200 nm to 230 nm.

11. The disinfecting lighting device of claim 1, further comprising a light-filtering medium which is used on the second light to produce a filtered light with a spectral power distribution (SPD) greater than 95% in a wavelength range of 200 nm to 230 nm.

12. The disinfecting lighting device of claim 11, wherein the light-filtering medium comprises an optical lens external to the second lens.

13. The disinfecting lighting device of claim 11, wherein the light-filtering medium comprises a light-filtering coating on a surface of the second lens.

14. The disinfecting lighting device of claim 11, wherein the light-filtering medium comprises a light-filtering coating on a surface of the second light source.

15. The disinfecting lighting device of claim 1, wherein the first light source comprises one or more light emitting diodes (LEDs).

16. The disinfecting lighting device of claim 1, wherein the first driver is dimmable and is capable of dimming the first light continuously.

17. The disinfecting lighting device of claim 1, wherein the second light source is replaceable without using any tool.

18. The disinfecting lighting device of claim 17, wherein the housing comprises an electric socket comprising G13, G5, R17D, FA8,2G11, or G24Q, and wherein the second light source has a corresponding electric base so the second light source is configured to be inserted into the electric socket on the housing without any tool.

19. The disinfecting lighting device of claim 1, wherein the first light source further comprises a third light source and a fourth light source, and wherein the disinfecting lighting device further comprises a circadian controller, wherein:
the housing further houses the circadian controller,
the third light source is configured to emit a third light with a spectral power distribution (SPD) less than 5% in a wavelength range of 410 nm to 490 nm,
the fourth light source is configured to emit a fourth light with a SPD greater than 15% in a wavelength range of 410 nm to 490 nm, and
the circadian controller is configured to mix the third light and the fourth light to generate a light output for the first means of lighting.

20. The disinfecting lighting device of claim 19, wherein an operation of the circadian controller is performed manually by a user.

21. The disinfecting lighting device of claim 19, wherein an operation of the circadian controller is performed automatically according to a circadian schedule.

22. The disinfecting lighting device of claim 21, wherein the circadian controller has a memory module configured to store the circadian schedule.

23. The disinfecting lighting device of claim 19, wherein each of the third light source and the fourth light source comprises one or more light emitting diodes (LEDs).
